(19) United States
(12) Patent Application Publication
(10) Pub. No.: US 2008/0262543 A1
(43) Pub. Date: Oct. 23, 2008

(54) Systems and methods for approximating surfaces

(75) Inventors: Mahalaxmi G. Bangera, Renton, WA (US); Edward S. Boyden, Cambridge, MA (US); Roderick A. Hyde, Redmond, WA (US); Muriel Y. Ishikawa, Livermore, CA (US); Edward K.Y. Jung, Bellevue, WA (US); Eric C. Leuthardt, St. Louis, MO (US); Dennis J. Rivet, St. Louis, MO (US); Michael A. Smith, Phoenix, AZ (US); Elizabeth A. Sweeney, Seattle, WA (US); Clarence T. Tegreene, Bellevue, WA (US); Lowell L. Wood, Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/805,617

(22) Filed: May 23, 2007

Related U.S. Application Data
(63) Continuation of application No. 11/788,767, filed on Apr. 19, 2007.

(51) Int. Cl. A61B 17/04

(52) U.S. Cl. 606/232

(57) ABSTRACT

Tissue is approximated by adhering anchors to opposing sides of a wound and coupling the anchors together.

### SUMMARY

In one aspect, a system for maintaining approximation of two surfaces includes a first anchor configured to adhere to a first surface that includes tissue, a second anchor configured to adhere to a second surface (which may be tissue or non-tissue), and an approximation mechanisms configured to operably link and maintain the first and second anchors in a predetermined spatial relationship. The approximation mechanisms may include a filament configured to maintain a tensile force tending to resist relative separation of the first and second anchors. The filament may be, for example, a suture (e.g., a shape-memory suture and/or a biodegradable suture), an elastomer, a cord, or a cable tie. The system may include a third anchor configured to adhere to a third surface, where the filament is configured to operably link and maintain the first, second, and third anchors in a predetermined relationship. The first and/or second anchors may be threaded onto the filament, and/or may include an opening configured to receive the filament (e.g., an eyelet, channel, and/or hook). The approximation mechanism may include a securing member configured to attach to each of the first and second anchors (e.g., a securing member configured to be inserted into openings in each of the first and second anchors, and/or a securing member including a conformable rod, a substantially straight rod, and/or a rod having a nonlinear shape). The approximation mechanism may include a stabilizing member (e.g., a flexible strip, a tape, and/or a flexible sheet) configured to support the first and second anchors in a predetermined relative position. The first and second anchors may be affixed to the stabilizing member, and/or may be configured to be coupled together via a coupling mechanism (e.g., a mechanism including a magnet, a mechanical fastener, an adhesive, and/or surfaces that adhere together by van der Waals forces). The approximation mechanism may be integral to at least one of the first and second anchors, such as a magnet that attaches the anchors together, a mechanical fastener (e.g., a tongue-in-groove connector, a retaining pin, a screw, a draw latch, a cable tie, and/or a hook and loop fastener) that fastens the anchors together reversibly or irreversibly, surfaces that adhere together by van der Waals forces, and/or an adhesive. The first anchor may be configured to adhere to the first surface via an adhesive, by penetrating tissue, by grasping, and/or by friction. Either or both of the anchors may include a surface-adherent portion configured to adhere to a surface and an engagement portion configured to engage the approximation mechanism. The surface-adherent portion and the engagement portion may be separate, separable, or integral, and may be configured to mechanically lock together, to be glued together with an adhesive, and/or to couple together with a fastener (e.g., a tongue-in-groove connector, a retaining pin, a screw, a draw latch, a cable tie, and/or a hook and loop fastener). The engagement portion may include an opening configured to accept at least a portion of the approximation mechanism, such as a loop, channel, and/or hook, and the approximation mechanism may include a filament configured to pass through the opening. Either or both of the anchors may include biocompatible material, biodegradable material, nonbiodegradable material, antimicrobial material, and/or material having a therapeutic property (e.g., a cell growth promoter, a cell growth inhibitor, a cytokine, a healing promoter, an antibiotic, a clotting modulator, an anti-inflammatory, and/or an anti-scarring agent). The system may include a third anchor configured to adhere to a third surface, wherein the approximation mechanism is configured to operably link and maintain the first, second, and third anchors in a predetermined spatial relationship. The second surface may include an organ for transplant, or an implant device such as a stent, a replacement heart valve, a breast implant, a cerebral stimulator, an endosseous implant, a bone growth matrix, an electrode, an aneurysm coil, and/or a graft.

In another aspect, a system for approximating body tissue includes a first anchor configured to adhere to body tissue, a second anchor configured to adhere to body tissue, and a traction member configured to draw the first anchor towards the second anchor. The first and second anchors and traction member are arranged to approximate body tissue upon drawing of the first anchor towards the second anchor. The traction member may be a suture, such as a shape-memory polymer suture, which may optionally be biodegradable. The anchors may be configured to adhere to tissue including, but not limited to, skin, blood vessel, muscle tissue, facial planes, dura, dermis, a visceral tube (e.g., an esophagus, a trachea, a respiratory tract, a stomach, a small intestine, a large intestine, a rectum, a ureter, and/or a vas deferens), and/or bone. The anchors may adhere by an adhesive, by penetrating tissue, and/or by friction. The first and second anchors may be configured to adhere to different body tissues. At least one of the first and second anchors may be configured to adhere by an adhesive, by penetrating tissue, and/or by friction. At least one of the anchors may include a tissue-adherent portion configured to adhere, to body tissue, and an engagement portion configured to engage the traction member for drawing together of the anchors, in which case the tissue-adherent portion and the engagement portion may be separate, separable, and/or integral. The engagement portion may include a channel, loop, and/or hook configured to accept the traction member. The engagement portion may be attached to the traction member, for example by being threaded on a traction member including a flexible cord such as a suture. The anchor may include a fastener (e.g., a tongue-in-groove connector, a retaining pin, a screw, a draw latch, a cable tie, and/or a hook and loop fastener) configured to couple together the tissue-adherent portion and the engagement portion, and/or the tissue-adherent portion and the engagement portion may be configured to mechanically lock together and/or to be glued together with an adhesive. The anchors may include a biocompatible material, a biodegradable material, a nonbiodegradable material, an antimicrobial material, and/or a material having a therapeutic property (e.g., a cell growth promoter, a cell growth inhibitor, a cytokine, a healing promoter, an antibiotic, a clotting modulator, an anti-inflammatory, and/or an anti-scarring agent).

In a further aspect, a system for approximating tissue includes a plurality of body-adherent tissue anchors and a securing member configured to hold the tissue anchors in a selected relative alignment. For example, the tissue anchors may each include an opening, and the securing member (e.g., a suture, a conformable rod, a substantially straight rod, and/or a rod having a nonlinear shape) may be configured to hold the tissue anchors by insertion of a portion of the member through each opening of the tissue anchors. The securing member may be configured to operably link to a first one of the plurality of body-adherent tissue anchors through a first connectivity mechanism, and to a second of the plurality of body-adherent tissue anchors through a second connectivity mechanism. (e.g., by insertion of at least a portion of a tissue anchor into an opening pointing the securing member, or via a mechanism such as a a tongue-in-groove connector, a retaining pin, a screw, a draw latch, a cable tie, and/or a hook and loop fastener). The tissue anchors may be configured to adhere to the body via an adhesive layer, by piercing, and/or by grasping.

In yet another aspect, a system for approximating tissue includes a stabilizing member (e.g., a flexible strip, tape, and/or flexible sheet) and a plurality of anchors configured to be attached to the stabilizing member in a selected relative arrangement. The stabilizing member and/or the plurality of anchors are configured to adhere to a body, and the anchors are configured to approximate tissue of the adhered body upon movement of selected ones of the anchors towards others of the anchors. The system may further include a traction member configured to draw the selected ones of the anchors towards others of the anchors, (such as those described herein, which may be biodegradable or nonbiodegradable). The plurality of anchors may be affixed to the stabilizing member. The anchors may be configured for pairwise attachment, for example via magnets, via mechanical fasteners (e.g., a tongue-in-groove connector, a retaining pin, a screw, a draw latch, a cable tie, and/or a hook and loop fastener), via an adhesive, and/or via van der Waals forces.

In still another aspect, a system for approximating tissue may include a plurality of tissue couplers that are configured to approximate attached tissue, and that are reversibly separable. The couplers may include magnets configured to attach the couplers together, and/or a mechanical fastener (e.g., a tongue-in-groove connector, a retaining pin, a screw, a draw latch, a cable tie, and/or a hook and loop fastener), which may be configured to irreversibly attach the couplers. The couplers may be configured to adhere to the body (e.g., via a body adhesive and/or by penetrating tissue), and/or they may be affixed to a stabilizing member (which may be configured to adhere to the body). The couplers may be configured to accept a tensioning member.

In yet still another aspect, a method of closing a wound includes adhering tissue anchors to tissue on opposing sides of the wound, and approximating the attached tissue by coupling the tissue anchors. Approximating the attached tissue may include bringing tissue on opposing sides of the wound into alignment, and coupling the anchors to maintain the alignment. At least a subset of the tissue anchors may include a tissue-adherent portion and a connector portion, and adhering each of the at least a subset of tissue anchors to tissue may include adhering the tissue-adherent portion to tissue, and connecting the connector portion to the tissue-adherent portion (e.g., before or after adhering the tissue-adherent portion to tissue). Coupling the tissue anchors may include slidably coupling the tissue anchors, groupwise coupling the tissue anchors, moving the tissue anchors relatively to within a magnetic coupling range, attaching the tissue anchors together with an adhesive, attaching the tissue anchors together with a hook and loop fastener, attaching the tissue anchors together via van der Waals forces, attaching the tissue anchors together with a mechanical fastener (e.g., a tongue-in-groove connector, a retaining pin, a screw, a draw latch, a cable tie, and/or a hook and loop fastener), pairwise coupling the tissue anchors, and/or coupling the tissue anchors with a common tensioning structure such as a suture (e.g., a shape-memory suture and/or a biodegradable suture) and/or an elastomer. Adhering tissue anchors may include attaching the tissue anchors to tissue on opposing sides of the wound with an adhesive, by piercing the tissue, and/or by grasping the tissue. The wound may be straight, curved, round, branched, stellate, and/or angled.

In yet a further aspect, a method of approximating a first region of body tissue and a second region of body tissue includes adhering a first tissue anchor to the first region of body tissue, adhering a second tissue anchor to the second region of body tissue, and approximating the adhered tissue by coupling the first and second tissue anchors. The first tissue anchor may include a tissue-adherent portion and a connector portion, and adhering the first tissue anchors to the first region of body tissue may include adhering the tissue-adherent portion to the first region of body tissue, and connecting the connector portion to the tissue-adherent portion (e.g., before or after adhering the tissue-adherent portion to the first region of body tissue). Coupling the first and second tissue anchors may include slidably coupling the first and second tissue anchors, moving the first and second tissue anchors relatively to within a magnetic coupling range, attaching the first and second tissue anchors together with an adhesive, attaching the first and second tissue anchors together with a hook and loop fastener, attaching the first and second tissue anchors together via van der Waals forces, attaching the first and second tissue anchors together with a mechanical fastener (e.g., a tongue-in-groove connector, a retaining pin, a screw, a draw latch, a cable tie, and/or a hook and loop fastener), and/or coupling the first and second tissue anchors with a common tensioning structure such as a suture (e.g., a shape-memory suture and/or a biodegradable suture) and/or an elastomer. Adhering the first tissue anchor may include attaching first the tissue anchor to the first region of body tissue with an adhesive, by piercing the tissue, and/or by grasping the tissue. The first and second regions of body tissue may have been separated before being approximated, and/or they may have been unconnected before being approximated. The method may further include cutting an incision between the first and second regions of body tissue before approximating the first and second regions of body tissue. Approximating the adhered tissue may include applying traction to at least one of the regions of body tissue.

In still a further aspect, a method of performing surgery includes adhering tissue couplers to tissue on opposing sides of a planned incision site for a body, cutting an incision between the adhered tissue couplers, accessing the interior of the body via the incision, and closing the incision by coupling the tissue couplers. Adhering the tissue couplers may include adhering a stabilizing member to the body, in which case the tissue couplers may be attached to the stabilizing member at the time that it is adhered to the body, or the method may further include attaching the tissue couplers to the stabilizing member. The stabilizing member may include fiducials that indicate a path for the planned incision. Cutting an incision may include making an opening in skin, in a blood vessel, in muscle tissue, in facial planes, in dura, in dermis, in a visceral tube (e.g., an esophagus, a trachea, a respiratory tract, a stomach, a small intestine, a large intestine, a rectum, a ureter, and/or a vas deferens), and/or in bone, and may include cutting with a tool such as a scalpel, cauter, trocar, needle, drill, curette, and/or laser. The incision may be straight, curved, round, branched, stellate, and/or angled. Accessing the interior of the body via the incision may include performing surgery in the body. Closing the incision may include applying a traction member such as a suture to the tissue anchors, coupling the tissue couplers manually, and/or coupling the tissue couplers automatically.

In yet still a further aspect, a method of preparing a body for surgery includes adhering tissue anchors to tissue on opposing sides of a planned incision site on a body. The tissue anchors are configured to be coupled in a configuration that approximates tissue. The tissue anchors may be attached to a stabilizer in a predetermined relative alignment. The stabilizer may include a fiducial that indicates a path for the planned incision. The stabilizer may adhere to the body, and may be a flexible sheet or a tape. The method may further include opening the body along the planned incision site, and may also include closing the body by coupling the tissue anchors.

In an additional aspect, a method of attaching an implant to body tissue includes attaching a first anchor to the body tissue, attaching a second anchor to the implant, and attaching the implant to the body tissue by coupling the first anchor and the second anchor. The first anchor may include a tissue-adherent portion and a connector portion, and attaching may include adhering the tissue-adherent portion to the body tissue, and connecting the connector portion to the tissue-adherent portion (e.g., before or after attaching the tissue-adherent portion to the body tissue). Coupling the first and second anchors may include slidably coupling the anchors, moving the anchors relatively to within a magnetic coupling range, attaching the anchors with an adhesive, attaching the anchors with a mechanical fastener (e.g., a tongue-in-groove connector, a retaining pin, a screw, a draw latch, a cable tie, and/or a hook and loop fastener), attaching the anchors via van der Waals forces, coupling the anchors with an approximation mechanism that joins the anchors in a defined spatial arrangement, such as a filament configured to maintain a tensile force tending to resist relative separation of the first and second anchors (e.g., a suture which may be optionally biodegradable and/or may contain a shape-memory material, an elastomer, a cord, and/or a cable tie). The first and second anchors may be threaded on the filament. The implant may include tissue, such as an organ for transplant, in which case the second anchor may optionally be attached before removal of the organ from a donor.

With respect to various embodiments described herein, the surfaces are tissue. It will be understood by those having skill in the art with knowledge of the present disclosure, that the systems and methods described herein can be utilized to approximate or maintain the approximation of two surfaces, at least one of which includes tissue. For ease of reading, the embodiments herein that describe two tissue surfaces are readily adaptable to approximating, or maintaining the approximation of, a tissue surface and a non-tissue surface. The various anchors described herein can be configured to adhere to non-tissue surfaces using the same or similar mechanisms that are described for adhering to tissue, for example by adhesive, surface penetration, friction and/or by any other means.

In addition to the systems and methods described herein for approximating or maintaining the approximation of two tissue surfaces surrounding an incision, it will be understood by those having skill in the art with knowledge of the present disclosure, that the systems and methods described herein can be utilized to approximate or maintain the approximation of surfaces surrounding any opening of tissue. Such an opening may be an incision or a wound. In addition, the systems and methods described herein may be used for approximating or maintaining the approximation of tissue surfaces which may be the same tissue type or they may be distinct. The tissues to be approximated may be naturally separated or may be separated due to an injury or other condition. It will be understood by those having skill in the art with knowledge of the present disclosure that the systems and methods described herein can be utilized to approximate, or maintain the approximation of, tissues such as, by way of non-limiting example, a blood vessel and a muscle tissue, or muscle tissue and bone tissue, ligament tissue to bone tissue, bone tissue to bone tissue, etc.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of an incision being closed with a suture and a set of tissue anchors.

FIG. 2 is a schematic of several different anchor embodiments.

FIG. 3 is a schematic of an incision being closed with a securing member and a set of tissue anchors.

FIG. 4 is a schematic of anchors arranged on a stabilizing member.

FIG. 5 is a schematic of several different embodiments of multi-part couplers.

FIG. 6 is a schematic of several different multi-part anchor embodiments.

FIG. 7 is a schematic of a two-part trocar for use in closing the fascia in a laparoscopic procedure.

FIG. 8 is a schematic of fascia being closed with a suture and a set of tissue anchors.

FIG. 9 is a flow chart of a method of closing a wound.

FIG. 10 is a flow chart of a method of performing surgery.

FIG. 11 is a flow chart of a method of preparing a body for surgery.

FIG. 12 is a schematic of a computer-implemented system for determining placement of tissue anchors.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

As used herein, the term “biocompatible” means a material the body generally accepts without a significant immune response/rejection or excessive fibrosis. In some embodiments, some immune response and/or fibrosis is desired. In other embodiments, vascularization is desired. In still other embodiments, vascularization is not desired. Biocompatible materials include, but are not limited to, synthetic organic materials such as clinically used nonbiodegradable and biodegradable and bioresorbable polymers including polyglycolide, optically active and racemic polylactides, polydioxanone, and polycaprolactone, polymers under clinical investigation including polyorthoester, polyanhydrides, and polyhydroxyalkanoate, early stage polymeric biomaterials including poly(lactic acid-co-lysine), and shape memory polymers (e.g., block copolymers of oligo(ε-caprolactone)diol and crystallisable oligo(ρ-dioxanone)diol, as described in Lendlein, et al., “Biodegradable, elastic shape-memory polymers for potential biomedical applications,” Science, 296(5573):1673-1676 (2002), which is incorporated by reference herein).

As used herein, “biodegradable” materials include materials that at least partially resorb into the body or otherwise break down over time, while “nonbiodegradable” materials include those that maintain substantial mechanical integrity over their lifetime in a body. Such “biodegradable” or “nonbiodegradable” materials are well known to those having skill in the art. In general, the anchors, couplers, traction members, securing members, tensioning members, stabilizing members, and other components described herein may be either biodegradable or nonbiodegradable, or may include both biodegradable and nonbiodegradable components. In some embodiments, these elements will be biocompatible, while in other embodiments, they may be partially or fully constructed from nonbiocompatible materials.

As used herein, “antimicrobial” materials include materials that have the capacity to inhibit the growth of or destroy pathogens, including but not limited to bacteria, fungi, and viruses. Such antimicrobial materials are well known to those having skill in the art and may include materials that are coated or impregnated with an antimicrobial agent or wherein the material itself possesses antimicrobial properties.

As used herein, a material having a “therapeutic property” is one that induces or facilitates a desired biological response. Materials having a therapeutic property are well known to those having skill in the art, and include, but are not limited to cell growth promoters, cell growth inhibitors, cytokines, healing promoters, antibiotics, clotting modulators, anti-inflammatories, and anti-scarring agents.

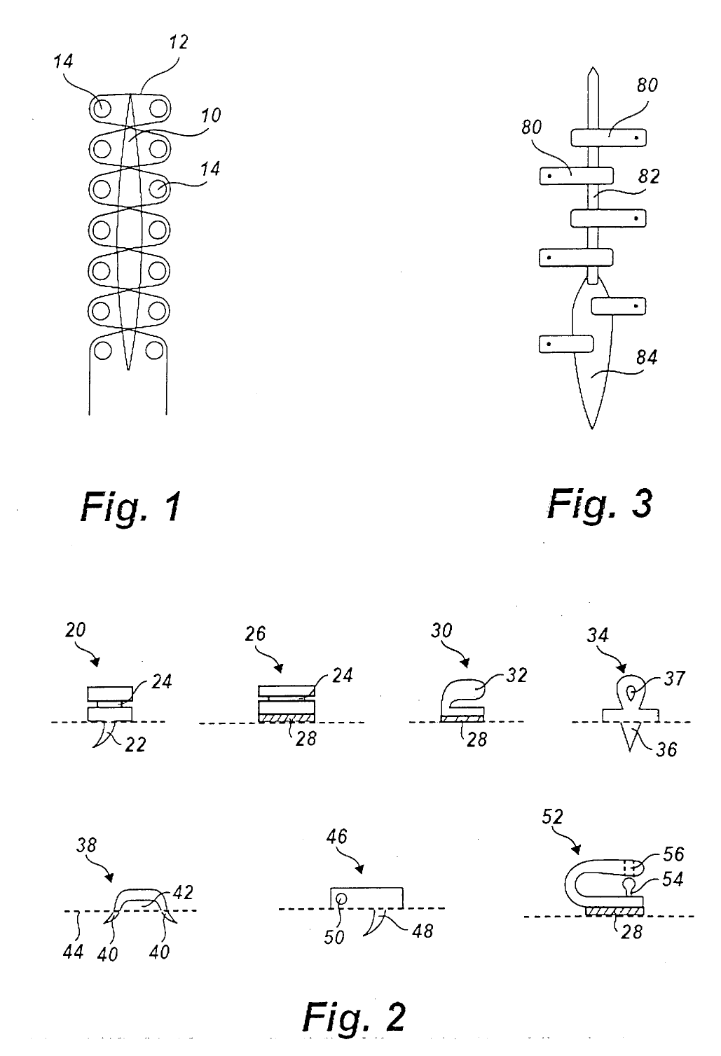

FIG. 1 illustrates an incision 10 being closed with a suture 12 and a set of tissue anchors 14. The tissue anchors 14 may be placed in the tissue before or after the incision 10 is made, and may be shaped to receive the suture 12. To close the incision 10, the suture 12 is wound around the anchors 14 as shown and pulled to tighten, approximating the body tissue in the region of the anchors 14. In some embodiments, the edges of the incision may be brought together by other means (e.g., manually by the surgeon), and the suture 12 may be used to maintain the approximation of the edges of the incision. Those of skill in the art of surgery will recognize that there are many possible patterns for placement of the anchors 14 and for winding of the suture 12, and will be able to select an appropriate configuration for any particular patient and incision. For example, the crossed suture 12 shown in FIG. 1 may not be desirable in all cases, and may be replaced by a suture winding that does not cross itself, such as a configuration (not shown) in which discrete sutures draw anchors 14 together pairwise across the incision 10, or a single suture arranged in a serpentine pattern. In other embodiments, it may not be desirable to place all anchors 14 at the same distance from incision 10, or to place the anchors 14 at regular intervals as shown in FIG. 1. For example, an irregular pattern or a pattern with localized concentrations of anchors 14 may be appropriate for locations having differential topographies, tissue types, expected movement ranges, stresses, or contact with surfaces, such as bandages, supports, clothing, or similar. The number and placement of the anchors 14 will also vary with the incision type, with fewer anchors 14 typically (but not always) being applied for smaller incisions. While FIG. 1 illustrates an incision 10 being closed, a similar arrangement may be used to close an accidental wound or to draw tissue into a desired configuration (e.g., in a face lift or other cosmetic procedure, or in a bladder suspension), or to attach tissue to an implanted device or other object (e.g., an organ for transplant) in a body. While FIG. 1 illustrates a straight incision 10, in other embodiments, the opening to be closed by the anchors may be curved, round, branched, stellate, and/or angled (e.g., in a sawtooth configuration).

FIG. 2 shows a variety of anchor configurations that may be used with a suture to close an incision as shown in FIG. 1. Anchor 20 includes a piercing structure 22 for placement in a body tissue, and a groove 24 to receive a suture. Anchor 26 also includes a groove 24 to receive a suture, but is adhered to the tissue via an adhesive layer 28. Anchor 30 is adhered to the tissue with an adhesive layer, and includes a hook 32 about which a suture may be looped. Anchor 34 includes a piercing structure 36 of a slightly different shape from that of anchor 20, and also includes an eyelet 37 through which a suture may be threaded. The piercing structure 36 may allow the anchor to be rotated, either manually or through the natural pulling action of a threaded suture. Anchor 38 includes two piercing prongs 40 like a staple, and creates an opening 42 through which a suture may be passed in cooperation with the underlying tissue 44. In some embodiments, this anchor may be pushed further into the tissue in a way that prevents movement of the suture, for example after the incision has been closed. Anchor 46 includes a piercing structure 48 and an eyelet 50, the eyelet 50 being disposed distal from the piercing structure 48 and along the surface of the body tissue. In some embodiments, the eyelets 50 of adjacent anchors 46 may be aligned as the tissue is closed. Anchor 52 includes a vertical post 54 and channel 56 allowing it to be snapped closed, for example after a suture has been threaded around it. In some embodiments, this closure may be reversible, while in others, it may be irreversible. In some embodiments, closure of the anchor 52 may restrict sliding of the suture, while in other embodiments, the suture may be able to slide through the anchor 52 after closure.

The specific structures of anchors shown in FIG. 2 shall not be interpreted to limit the shape or design of the anchors described and claimed herein. By way of non-limiting example, the piercing structure 22 of anchor 20 may be used in place of the adhesive layer 28 shown with anchor 30; the eyelet 50 of anchor 46 may be used with the adhesive layer 28 shown with anchor 30 or with the piercing structure 36 shown with anchor 34. Various combinations of the piercing structures shown, as well as those not shown but known to those of skill in the art, can be used with any suture-holding structure or any other securing member or mechanism.

FIG. 3 shows another embodiment, in which anchors 80 are secured via insertion of a securing member 82, which is a conformable rod in the illustrated embodiment. The illustrated anchors 80 are similar to the anchors 46 of FIG. 2, including a piercing structure (not visible in FIG. 3) and an eyelet through which conformable rod 82 may be inserted. As shown, the rod 82 is partially inserted through the anchors 80, so that the incision 84 is partially closed. In other embodiments, the anchors 80 or the securing member 82 may include other attachment structures, such as hooks, mating surfaces (to which adhesive may optionally be applied), and/or mechanical fasteners (e.g., hook and loop fasteners, draw latches, screws, etc.). By way of non-limiting example, securing member 82 may include a series of hooks configured to receive anchors 80; anchors 80 may include hooks configured to attach to securing member 82 (which may optionally include predetermined attachment points for anchors 80); securing member 82 may include snap fittings into which mating portions of anchors 80 may be inserted; anchors 80 and securing member 82 may include holes or other areas configured for attachment of screws or other fasteners that secure anchors 80 and securing member 82 together. In embodiments in which the securing member 82 is conformable, it may be conformed to match the shape of an incision, or it may be conformed before or after insertion in order to apply a mechanical force to tissue in order to reshape it (e.g., in cosmetic surgery). In some embodiments, the process of inserting securing member 82 may bring the anchors 80 together, while in other embodiments, the edges of the incision 84 may be brought into alignment before the securing member 82 is deployed.

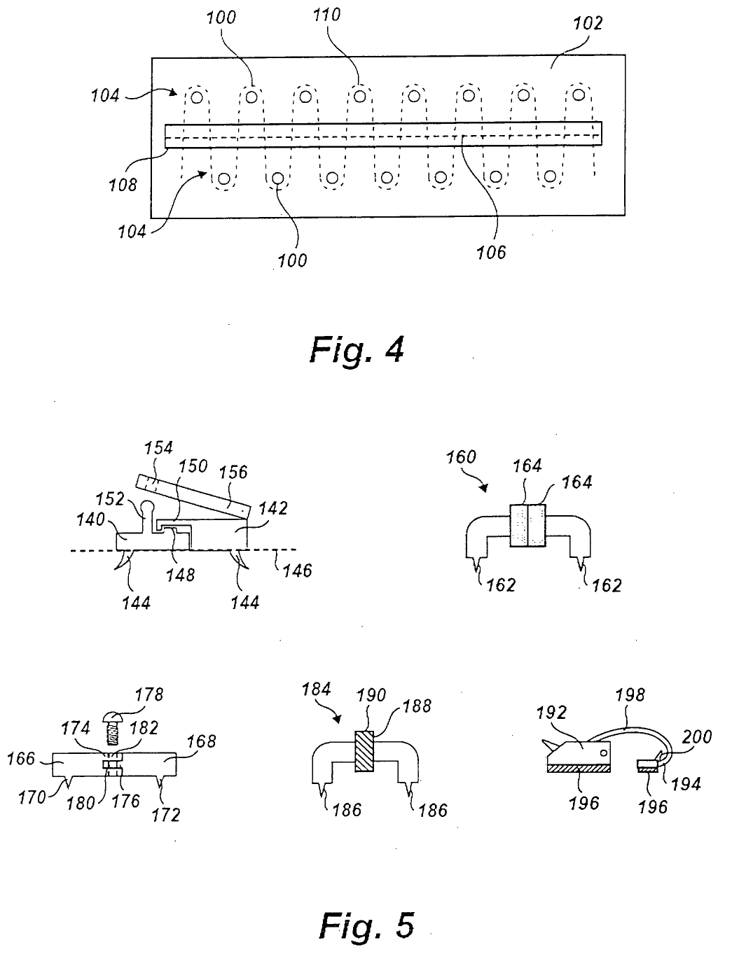

FIG. 4 is a schematic of anchors 100 arranged on a stabilizing member 102. In the embodiment shown, the stabilizing member 102 includes a flexible tape base designed to adhere to the tissue of interest. The anchors are arranged in parallel rows 104 on opposite sides of a planned incision site 106. In some embodiments, the flexible tape base may be placed on the patient prior to making the incision. The illustrated embodiment includes an opening 108 along the planned incision site 106, but other embodiments may omit the opening. The anchors 100 may adhere to the stabilizing member 102, which in turn adheres to the tissue of interest, via an adhesive, or they may include mechanical fasteners or other structures to facilitate their attachment to tissue (e.g., piercing structures such as those shown in anchors 20, 34, 38, 46 of FIG. 2). In one method of use, the stabilizing member 102 is placed on the body with opening 108 positioned at the planned incision site 106. The incision is made, and surgery is performed on the body via the incision. At the conclusion of the surgery, a suture is threaded around the anchors 100 along serpentine path 110, and tightened to draw the anchors 100 together, thereby closing the incision (in some embodiments, the incision may be closed by other means, and the suture may maintain the closure). In other embodiments, opening 108 may be omitted, and the incision performed through the stabilizing member 102, or the stabilizing member 102 may be placed after the incision is made (e.g., after the surgery is completed). In some embodiments, the stabilizing member 102 may be applied to a wound (e.g., an accidental wound). Rather than a suture, the incision may be closed by application of a securing member as described above in connection with FIG. 3, or by direct connection of couplers as described below in connection with FIG. 5. The stabilizing member 102 may be placed on the skin, or on other tissue such as muscular or vascular tissue.

FIG. 5 shows several different embodiments of couplers that may be connected without the use of a tensioning member or a securing member as described above. In some embodiments, a specialized or general purpose tool may be used to connect anchors together. Couplers 140, 142 include piercing structures 144 that secure the couplers to underlying tissue 146. Coupler 140 includes a temporary alignment pin 148 configured to mate with a corresponding alignment groove 150 on coupler 142. In addition, coupler 140 includes a permanent (or, optionally, semipermanent) retaining pin 152 configured to mate with a channel 154 in hinged connector 156 on coupler 142. In one method of use, the couplers 140, 142 may be secured to tissue with temporary alignment structures 148, 150 connected. The temporary alignment structures 148, 150 may then be disconnected to permit access to an incision site, for example, to open an incision after the couplers 140, 142 have been placed. Upon closing, both temporary alignment structures 148, 150 and permanent retaining structures 152, 154 may be connected, permanently (or, optionally, semipermanently) closing the incision while maintaining the alignment of underlying tissue.

Couplers 160 include piercing structures 162, and permanent magnets 164. In use, these couplers may be placed on either side of a wound or a planned incision, and optionally rotated to increase the distance between permanent magnets 164 during access to the wound. Upon closing, the couplers 160 may be rotated (if necessary) to align the magnets, and brought into proximity to magnetically adhere them together, securing the underlying tissue. Couplers 166, 168 include piercing structures 170, 172 for securing them to tissue. A groove 174 in coupler 166 mates with a tongue 176 in coupler 168 to couple the couplers. This connection can be reversibly or irreversibly secured by insertion of a screw 178 through channels 180, 182 in the couplers 166, 168. Couplers 184 include piercing structures 186, and matable surfaces 188. In use, these couplers may be placed on either side of a wound or a planned incision, and optionally rotated to orient the matable surfaces away from the work area. Upon closing, the couplers 184 may be rotated (if necessary) to align the matable surfaces, which may then be secured together with adhesive 190. Couplers 192, 194 include adhesive 196 for attachment to tissue (or to a stabilizing member, not shown, or other mechanism for attachment to tissue). Coupler 192 includes latch arm 198, which engages keeper 200 on coupler 194 to form a draw latch assembly. Latch arm 198 may be rotated away from the work area during surgery, and subsequently engaged to close an underlying incision.

While the couplers illustrated in FIG. 5 are generally illustrated for coupling in pairwise configurations, in other embodiments, couplers may cooperate in larger groups to close incisions or other wounds. For example, couplers may be arranged in a “zipper” configuration to close a wound along its length. Such an arrangement may include a specialized or general-purpose coupling tool (e.g., a zipper pull) to connect couplers together and/or to separate them.

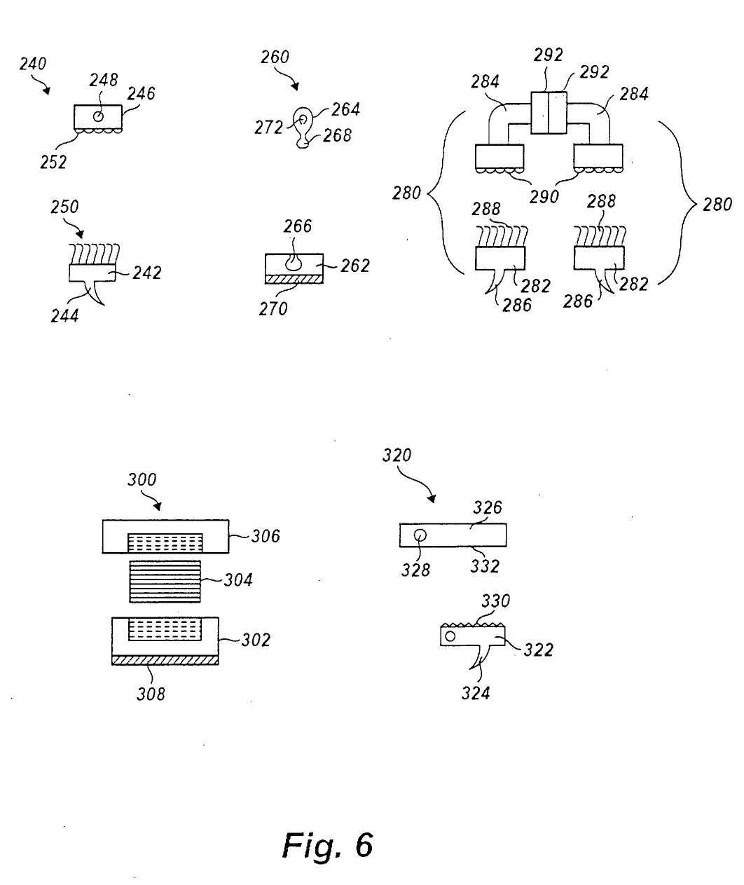

FIG. 6 is a schematic of several different multi-part anchor embodiments. Each embodiment includes a portion that adheres to tissue, and a portion that engages a suture, a stabilizing member, another anchor, or another closing mechanism. Anchor 240 includes a tissue adherent portion 242, which is configured to adhere to tissue via piercing mechanism 244, and connector portion 246, which is configured to engage a suture via opening 248. The tissue adherent portion 242 and the connector portion 246 are configured to be connected together via hook-and-loop fasteners 250, 252 (e.g., VELCRO™). Anchor 260 includes a tissue adherent portion 262 and a connector portion 264, which are configured to snap together via mechanical fasteners 266, 268. Tissue adherent portion 262 includes an adhesive layer 270 configured to adhere to tissue. Connector 264 includes an eyelet 272 configured to receive a suture (not shown). In some embodiments, mechanical fasteners 266, 268 may be configured to form a rotatable connection, which may facilitate alignment of a suture. In either embodiment of anchors 240 or 260, connector portions 246 or 264 may optionally be pre-threaded onto a suture or a stabilizing member before they are connected to their respective tissue adherent portions 242 or 262, or they may be connected to their respective tissue adherent portions 242 or 262 and subsequently threaded with a suture or stabilizing member.

Anchors 280 each include a tissue adherent portion 282 and a connector portion 284. The tissue adherent portions 282 are configured to adhere to tissue via piercing structures 286. Connector portions 284 are configured to attach to tissue adherent portions 282 via hook-and-loop fasteners 288 and 290 (e.g., VELCRO™). Connector portions 284 are also configured to engage one another via magnets 292. In one method of use, tissue adherent portions 282 may be placed on opposing sides of an incision site, before or after cutting the incision. Upon closing, connectors 284 may be connected to tissue adherent portions 282 and their respective magnets 292 engaged (before or after connection to tissue adherent portions 282), thereby closing the incision.

Anchor 300 is a three-part anchor, including a tissue adherent portion 302, a first connector portion 304 configured to screw into tissue adherent portion 302, and a second connector portion 306 configured to screw onto connector portion 304. In one method of use, a plurality of tissue adherent portions 302 are adhered to tissue via adhesive layers 30.8, for example before an incision is made in the tissue. When it is desired to close the opening, first connector portions 304 are screwed into each respective tissue adherent portion 302. At this point, a suture or other tensioning member (not shown) may be wound about connector portions 304. In other embodiments, second connector portions 306 may be partially or fully screwed onto their respective first connector portions 304 before winding or before tightening of the tensioning member. In some embodiments, once the tensioning member has been tightened sufficiently to close the incision, second connector portions 306 may be further screwed onto first connector portions 304, thereby clamping the tensioning member between tissue adherent portions 302 and second connector portions 306, thereby inhibiting further movement of the tensioning member.

Anchor 320 includes tissue adherent portion 322, which adheres to tissue via piercing structure 324, and connector portion 326, which includes eyelet 328. Tissue adherent portion 322 and connector portion 326 are configured to attach to one another via van der Waals forces. In the illustrated embodiment, surface 330 includes nanotubes that adhere to flat surface 332 when they are placed in contact (see, e.g., Yurdumakan, et al., “Synthetic gecko foot-hairs from multiwalled carbon nanotubes,” Chem. Commun., 2005:3799-3801, which is incorporated by reference herein). In this embodiment, eyelet 328 is located at a distal end of tissue adherent portion 322 when tissue adherent portion 322 and connector portion 326 are attached together. In some embodiments, a straight (or shaped) stabilizing element (not shown) may be threaded through eyelets 328 of a plurality of anchors 320 on opposing sides of a wound, for example in the configuration illustrated in FIG. 3.

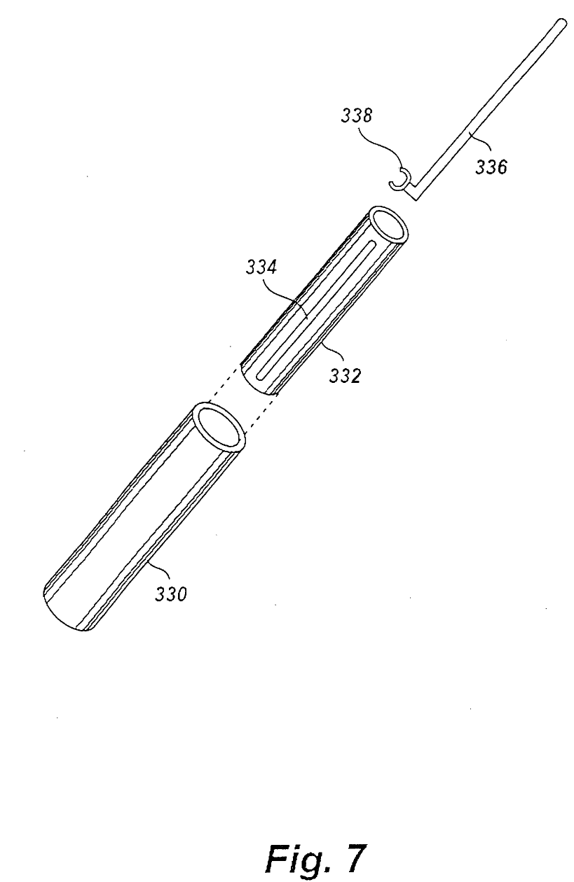

FIG. 7 illustrates a two-part trocar for use in laparoscopic procedures. The trocar includes a first cylinder 330 having solid walls, and a second cylinder 332 having one or more longitudinal slots 334. As shown, the second cylinder 332 is sized to fit snugly within first cylinder 330. In other embodiments, the outer diameter of second cylinder 332 may be smaller than the inner diameter of first cylinder 330, producing a loose fit between the cylinders. In still other embodiments, the second cylinder 332 may be sized to fit over first cylinder 330, with either a loose or a snug fit. In still other embodiments, the first cylinder 330 may be eliminated. In such embodiments, if it is necessary to insufflate the underlying body cavity, it may be desirable that a mechanism for sealing slots 334 be integrated into second cylinder 332 in order to maintain pressure within the cavity.

In one method of use, first cylinder 330 is inserted into a body cavity (e.g., the abdominal cavity), using a round cutter (not shown) to penetrate the cavity wall. Second cylinder 332 may be integral with first cylinder 330 during insertion, or may be inserted into (or around) first cylinder 330 previously or subsequently, either before or after a laparoscopic procedure is performed. For example, the first cylinder 330 may be inserted as a conventional trocar, and a laparoscopic procedure may be performed. Subsequent to the procedure, but before closing, second cylinder 332 is then inserted into first cylinder 330, and first cylinder 330 is fully or partially retracted from the body. An anchor placement device 336, loaded with anchor 338 is then inserted into second cylinder 332. As shown, the anchor is a split ring, but any of the anchor configurations described herein may be used. In the illustrated embodiment, anchor 338 includes a shape memory alloy. The anchor 338 is inserted through the slot 334 to contact opposing sides of the fascia, and the shape memory phase change is triggered (e.g., by local heating), closing the split ring and piercing the fascia. Multiple anchors 338 may be placed, either using multiple slots 334 or by rotating second cylinder 332 in order to access different positions along the circumference of the fascial opening. Once the anchors 338 have been placed, second cylinder 332 may be fully or partially withdrawn from the opening.

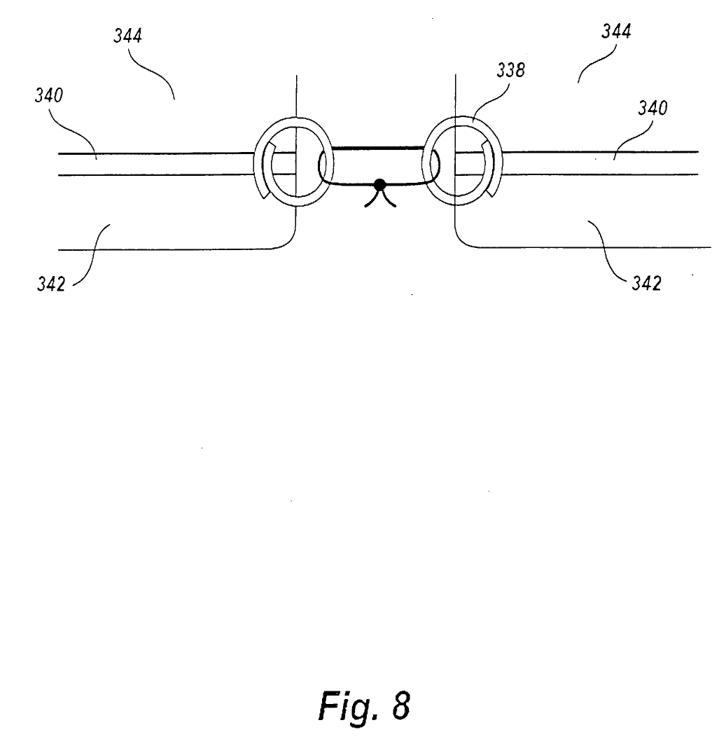

FIG. 8 illustrates two split ring anchors 338 which have pierced the fascia 340 on either side of a round laparoscopic incision. As shown, the anchors 338 also at least partially penetrate peritoneum 342 and fatty tissue 344. Anchors 338 are connected by a suture 346. The suture may be threaded before or after removal of cylinders 330, 332. Tension may be applied to suture 346 to close the fascia, for example after cylinders 330, 332 have been removed from the incision. In the illustrated embodiment, the suture connects two anchors 338 on opposing sides of the incision, but it will be understood that more anchors may be connected, either by a single suture or other connector looped through all of them, or by a series of pairwise connections (or other connections of smaller subsets of the placed anchors). Tissue anchors may be analogously used to close other layers such as the peritoneum, the muscle layers, and/or the skin. While split-ring anchors 338 have been illustrated in FIG. 7 and FIG. 8, other anchor configurations may be more or less desirable for any particular tissue type and geometry. In some configurations, a suture or other tensioning device may be prethreaded onto tissue anchors, or the anchors may be configured to couple to one another without use of a tensioning device.

In general, the anchors, couplers, traction members, securing members, tensioning members, stabilizing members, and other components described herein may be adjustable or selectively controlled, for example to loosen tension as a joint heals and becomes more flexible or to permit expansion of skin prior to reconstructive surgery or removal for a graft. In particular, any of these components may form a part of or be configured to cooperate with the adjustable implants described in co-pending and commonly owned U.S. application Ser. Nos. 11/710,591, filed Feb. 22, 2007 and entitled, “CODED-SEQUENCE ACTIVATION OF SURGICAL IMPLANTS,” and Ser. No. 11/710,592, filed Feb. 22, 2007 and entitled, “CODED-SEQUENCE ACTIVATION OF SURGICAL IMPLANTS,” both of which are incorporated by reference herein. Any of these components may also be controllable by changing shape or conformation so that such change results in the approximation of surfaces attached to selected anchors, for example via the use of temperature-sensitive, light-sensitive (e.g., ultraviolet light-sensitive), touch-sensitive, elastomeric (e.g., an elastomer that is configured to secure each anchor and can reconfigure in a way to approximate surfaces attached to the anchors), or remotely controllable mechanisms.

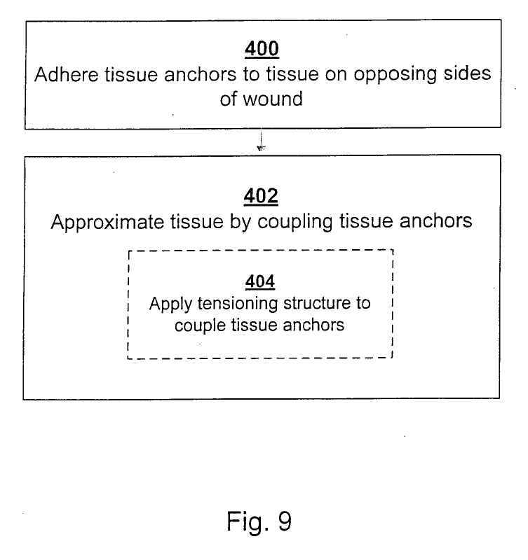

FIG. 9 is a flow chart illustrating a method of closing a wound. The method includes adhering tissue anchors (e.g., anchors such as but not limited to those described in FIG. 2, FIG. 5, and/or FIG. 6) to tissue on opposing sides of a wound, 400, and approximating the tissue by coupling the tissue anchors, 402. For example, the tissue anchors may be coupled via a tensioning element such as a suture, 404.

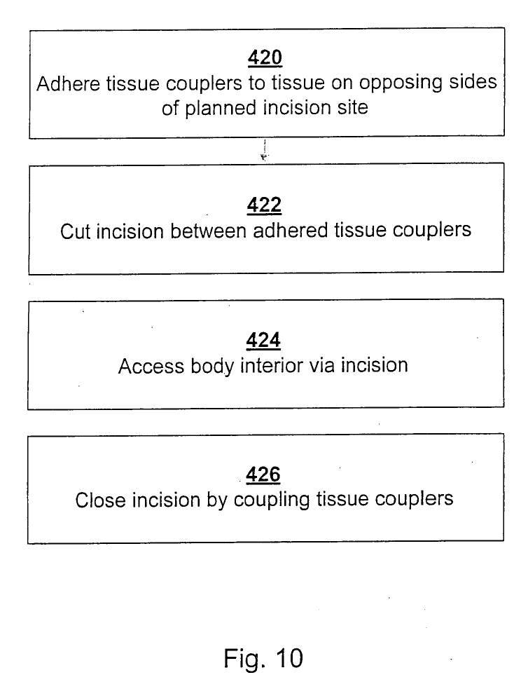

FIG. 10 is a flow chart illustrating a method of performing surgery. The method includes adhering tissue couplers (e.g., couplers such as but not limited to those described in FIG. 2, FIG. 5, and/or. FIG. 6) to tissue on opposing sides of a planned incision site, 420, cutting an incision between the adhered tissue couplers, 422, accessing the interior of the body via the incision (e.g., to perform a surgical procedure), 424, and closing the incision by coupling the tissue couplers, 426. The incision may be, for example, a straight incision, a curved incision, or a round incision (e.g., a round cut such as that made by a trocar). In some embodiments, couplers may be coupled together manually, while in other embodiments, couplers may be coupled together automatically. In some embodiments, the surgery may be endoscopic.

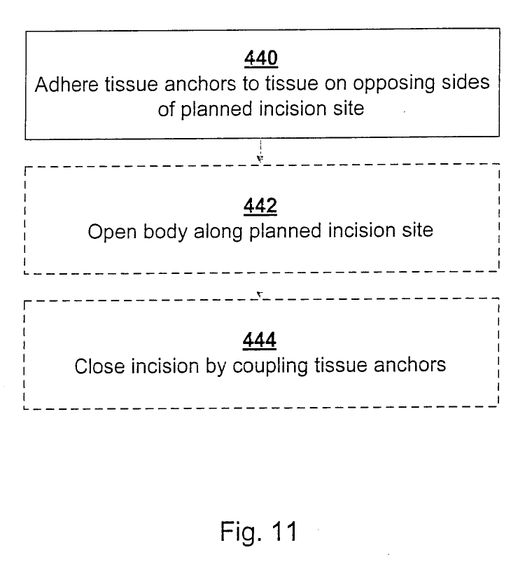

FIG. 11 is a flow chart illustrating a method of preparing a body for surgery. The method includes adhering tissue anchors to tissue, 440, on opposing sides of a planned incision site. The method may optionally also include opening the body along the planned incision site, 442, and/or closing the incision by coupling the tissue anchors, 444. The incision may be, for example, a straight incision, a curved incision, or a round incision (e.g., a round cut such as that made by a trocar).

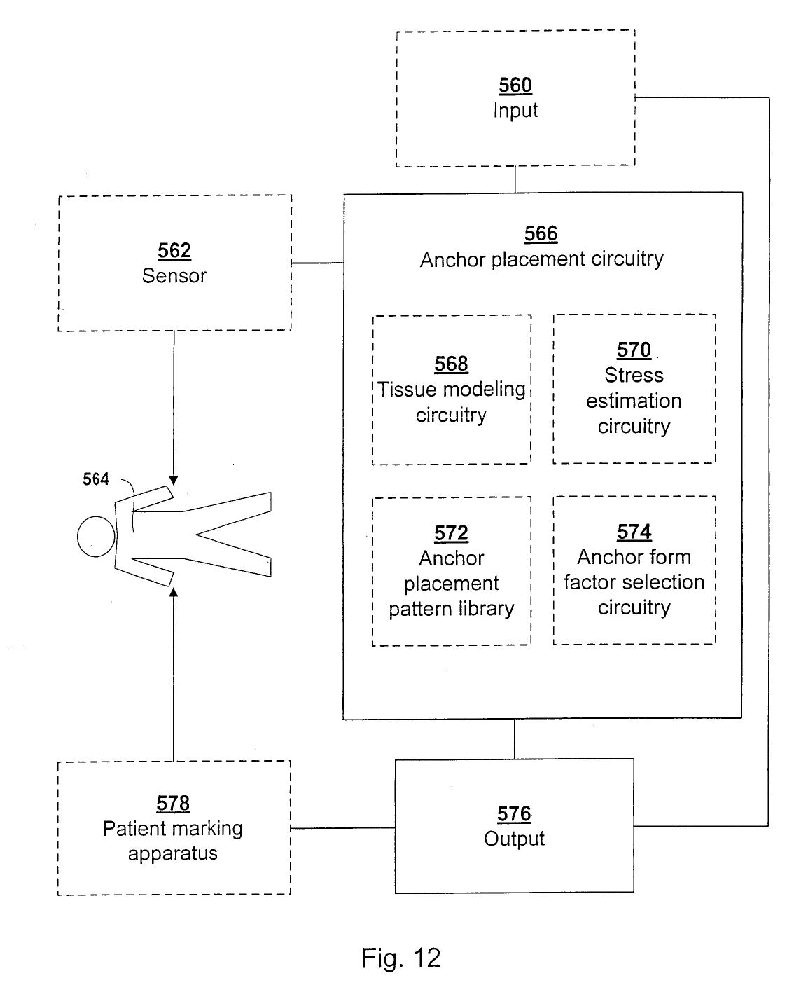

FIG. 12 illustrates a system for determining placement of tissue anchors (or other suture attachments) for closing an incision. The system may include an input device 560 (e.g., a mouse, keyboard, touchscreen, or other machine input system), configured to allow a surgeon to specify a surgery type and/or an incision location. It may further include a sensor 562 that measures one or more physiological parameters of a patient 564 upon whom surgery will be performed. For example, the sensor 562 may include an imaging device that maps the position of organs or other physiological structures that may be taken into account in closing an incision, or it may be a reader (e.g., an optical reader) that senses a planned incision location that a surgeon has marked on the body of patient 564. The input device 560 and/or the sensor 562 may communicate information about the body of patient 564 and/or about the planned surgery to anchor placement circuitry 566. Anchor placement circuitry 566 may include various subcircuits or subroutines, including but not limited to tissue modeling circuitry 568, stress estimation circuitry 570, anchor placement pattern library 572, and/or anchor form factor selection circuitry 574.

Tissue modeling circuitry 568 may include circuitry configured to build a computer-based model (e.g., a finite element model and/or an analytical model) of the tissue of the patient 564, for example including specific measurements of sensor 562 and/or physiological or other parameters specified using input device 560. This computer-based model may be used to determine suggested placement for tissue anchors, for example by calculation of the expected response of tissue to particular anchor configurations, and/or by application of stored heuristic rules for expected tissue response. Stress estimation circuitry 570 may be configured to determine expected stresses on anchors and/or on tissue for particular anchor configurations, or it may include optimizing circuitry designed to determine an optimum anchor configuration for a specified design goal. Anchor placement pattern library 372 may include stored configurations of anchors that have been specified by an operator, previously calculated, or otherwise determined. Other portions of the anchor placement circuitry 566 (e.g. tissue modeling circuitry 568 and/or stress estimation circuitry 570) may use the anchor placement pattern library 572 to generate initial placement patterns for calculation, including as a starting point for optimization routines. Anchor form factor selection circuitry 574 may store information about the different form factors of different anchors (such as but not limited to those described herein, e.g., in FIG. 2, FIG. 5, and/or FIG. 6), and may further include information about available sizes and mechanical performance of different anchors. It may further include circuitry configured to select a suggested anchor or group of anchors for the particular surgery planned for patient 564.

The system further includes an output device 576 (e.g., a monitor, a printer, a bar code printer, and/or a controller for a patient marking apparatus 578), which may produce a machine-readable and/or a human-readable output. This output may include calculated anchor placement patterns, tissue responses, anchor stresses, anchor form factors, or other data relevant for placement of anchors during surgery. Output may be iterative and/or interactive, so that a user specifying input via input device 560 may modify input or specify additional inputs in response to output received via output device 576. For example, output device 576 may output a selection of anchor placement patterns from anchor placement pattern library 572, and a user may select from among these patterns using input device 560. Once an anchor placement pattern has been established by anchor placement circuitry 566, output device 576 may pass data and/or control instructions to a patient marking device 578, which may temporarily or permanently mark desired anchor placement directly on the patient 564, or on a tape or other stabilizing member configured to maintain relative anchor locations for attachment to the patient 564. In other embodiments, the patient marking device may actually place anchors on a stabilizing member for application to a patient 564.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those having skill in the art will recognize that the state of the art of circuit design has progressed to the point where there is typically little distinction left between hardware and software implementations of aspects of systems. The use of hardware or software is generally a design choice representing tradeoffs between cost, efficiency, flexibility, and other implementation considerations. Those having skill in the art will appreciate that there are various vehicles by which processes, systems and/or other technologies involving the use of logic and/or circuits can be effected (e.g., hardware, software, and/or firmware), and that the preferred vehicle will vary with the context in which the processes, systems and/or other technologies are deployed. For example, if an implementer determines that speed is paramount, the implementer may opt for a mainly hardware and/or firmware vehicle. Alternatively, if flexibility is paramount, the implementer may opt for a mainly software implementation. In these or other situations, the implementer may also opt for some combination of hardware, software, and/or firmware. Hence, there are several possible vehicles by which the processes, devices and/or other technologies involving logic and/or circuits described herein may be effected, none of which is inherently superior to the other. Those skilled in the art will recognize that optical aspects of implementations may require optically-oriented hardware, software, and or firmware.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “an anchor” should typically be interpreted to mean “at least one anchor”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two anchors,” or “a plurality of anchors,” without other modifiers, typically means at least two anchors). Furthermore, in those instances where a phrase such as “at least one of A, B, and C,” “at least one of A, B, or C,” or “an [item] selected from the group consisting of A, B, and C,” is used, in general such a construction is intended in the sense one having skill in the art would understand the convention (e.g., any of these phrases would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1.-61. (canceled)

62. A system for approximating body tissue, comprising:
a first anchor configured to adhere to body tissue;
a second anchor configured to adhere to body tissue; and
a traction member configured to draw the first anchor towards the second anchor, the first and second anchors and traction member being arranged to approximate body tissue upon drawing of the first anchor towards the second anchor.

63. The system of claim 62, wherein the traction member includes a suture.

64. (canceled)

65. (canceled)

66. The system of claim 62, wherein at least one of the first and second anchors is configured to adhere to skin.

67. The system of claim 62, wherein at least one of the first and second anchors is configured to adhere to a blood vessel.

68. The system of claim 62, wherein at least one of the first and second anchors is configured to adhere to muscle tissue.

69. The system of claim 62, wherein at least one of the first and second anchors is configured to adhere to a facial plane.

70. The system of claim 62, wherein at least one of the first and second anchors is configured to adhere to dura.

71. The system of claim 62, wherein at least one of the first and second anchors is configured to adhere to dermis.

72. The system of claim 62, wherein at least one of the first and second anchors is configured to adhere to a visceral tube.

73. (canceled)

74. The system of claim 62, wherein at least one of the first and second anchors is configured to adhere to bone.

75. The system of claim 62, wherein the first anchor and the second anchor are configured to adhere to different body tissues.

76.-78. (canceled)

79. The system of claim 62, wherein at least one of the first and second anchors includes:
a tissue-adherent portion configured to adhere to body tissue; and
an engagement portion configured to engage the traction member for drawing together of the first and second anchors.

80. The system of claim 79, wherein the tissue-adherent portion and the engagement portion are separate.

81. The system of claim 79, wherein the tissue-adherent portion and the engagement portion are separable.

82. The system of claim 79, wherein the tissue-adherent portion and the engagement portion are integral.

83.-92. (canceled)

93. The system of claim 62, wherein at least one of the first and second anchors includes a biocompatible material.

94.-96. (canceled)

97. The system of claim 62, wherein at least one of the first and second anchors includes a material having a therapeutic property.

98. (canceled)

99. A system for approximating tissue, comprising:
a plurality of body-adherent tissue anchors; and
a securing member configured to hold the tissue anchors in a selected relative alignment.

100. The system of claim 99, wherein the tissue anchors each comprise an opening, and wherein the securing member is configured to hold the tissue anchor by insertion of a portion of the member through each opening of the tissue anchors.

101.-104. (canceled)

105. The system of claim 99, wherein the securing member is configured to operably link to a first one of the plurality of body-adherent tissue anchors through a first connectivity mechanism, and to operably link to a second one of the plurality of body-adherent tissue anchors through a second connectivity mechanism.

106.-110. (canceled)

111. A system for approximating tissue, comprising:
a stabilizing member; and
a plurality of anchors configured to be attached to the stabilizing member in a selected relative arrangement, wherein at least one of the stabilizing member and the plurality of anchors is configured to adhere to a body,
wherein the anchors are configured to approximate tissue of the adhered body upon movement of selected ones of the anchors towards others of the anchors.

112. The system of claim 111, further comprising a traction member configured to draw the selected ones of the anchors towards the others of the anchors.

113.-116. (canceled)

117. The system of claim 111, wherein the plurality of anchors are affixed to the stabilizing member.

118. The system of claim 111, wherein the stabilizing member is a flexible strip.

119.-120. (canceled)

121. The system of claim 111, wherein the anchors are configured for pairwise attachment.

122.-131. (canceled)

132. A system for approximating tissue, comprising:
a plurality of tissue couplers, wherein the couplers are configured to approximate attached tissue, and wherein the couplers are reversibly separable.

133. The system of claim 132, wherein the couplers include magnets configured to attach the couplers together.

134. The system of claim 132, wherein the couplers include a mechanical fastener configured to connect the couplers.

135.-141. (canceled)

142. The system of claim 132, wherein the couplers are configured to adhere to the body.

143.-147. (canceled)

148. A method of closing a wound, comprising:
adhering tissue anchors to tissue on opposing sides of the wound; and
approximating the attached tissue by coupling the tissue anchors.

149. The method of claim 148, wherein approximating the attached tissue includes:
bringing the tissue on opposing sides of the wound into alignment; and
coupling the tissue anchors to maintain the alignment.

150. (canceled)

151. The method of claim 148, wherein at least a subset of the tissue anchors include a tissue-adherent portion and a connector portion, and wherein adhering each of the at least a subset of the tissue anchors to tissue includes:
adhering the tissue-adherent portion to tissue; and
connecting the connector portion to the tissue-adherent portion.

152.-154. (canceled)

155. The method of claim 148, wherein coupling the tissue anchors includes moving the tissue anchors relatively to within a magnetic coupling range.

156. The method of claim 148, wherein coupling the tissue anchors includes attaching the tissue anchors together with an adhesive.

157. The method of claim 148, wherein coupling the tissue anchors includes attaching the tissue anchors together with a hook and loop fastener.

158. The method of claim 148, wherein coupling the tissue anchors includes attaching the tissue anchors together via van der Waals forces.

159. The method of claim 148, wherein coupling the tissue anchors includes attaching the tissue anchors together with a mechanical fastener.

160.-161. (canceled)

162. The method of claim 148, wherein coupling the tissue anchors comprises coupling the tissue anchors with a common tensioning structure.

163.-170. (canceled)

171. The method of claim 148, wherein the wound is curved.

172. The method of claim 148, wherein the wound is round.

173.-243. (canceled)
